(19) United States
(12) Patent Application Publication
(10) Pub. No.: US 2018/0021237 A1
(43) Pub. Date: Jan. 25, 2018

(54) ADHESIVE MATERIALS AND SEQUESTERED CURING AGENTS USED TO PRODUCE THEM

(71) Applicants: Hans P.I. Claesson, Lexington, MA (US); Laurence J Berlowitz, Beverly, MA (US); Matthew Fullana, Wilmington, MA (US); Gary E Wnek, Cleveland, OH (US)

(72) Inventors: Hans P.I. Claesson, Lexington, MA (US); Laurence J Berlowitz, Beverly, MA (US); Matthew Fullana, Wilmington, MA (US); Gary E Wnek, Cleveland, OH (US)

(21) Appl. No.: 15/650,920

(22) Filed: Jul. 16, 2017

Related U.S. Application Data
(60) Provisional application No. 62/364,560, filed on Jul. 20, 2016.

(51) Int. Cl. A61K 8/65; C07K 14/78; A61K 47/60; A61K 38/01

(52) U.S. Cl. CPC A61K 8/65; A61K 38/014; C07K 14/78; A61K 47/60; C08G 65/34

(57) ABSTRACT

Certain configurations of adhesive materials are described which comprise a crosslinked derivatized atelocollagen that can be cured with a sequestered curing agent. In some configurations, the crosslinked, derivatized atelocollagen is used to provide an adhesive that can be used to hold two or more tissues to each other for some period and permit a tissue repair process to occur.

### TECHNOLOGICAL FIELD

Certain features, aspects and embodiments are directed to adhesive materials and methods of producing and using them. In particular, certain embodiments are directed to bioadhesives that can be produced using sequestered curing agents to increase the overall adhesive nature of the bioadhesives.

### SUMMARY

Certain features, aspects and embodiments described herein are directed to bioadhesive materials which comprise atelocollagen that has been derivatized and/or cross-linked in combination with a sequestered curing agent to provide an adhesive material.

In a first aspect, an adhesive material comprises a crosslinked, derivatized atelocollagen and a sequestered curing agent.

In another aspect, an adhesive material comprises a crosslinked, derivatized atelocollagen that has been cross-linked in the presence of at least two different functionalized crosslinking agents and an encapsulated curing agent.

In an additional aspect, an adhesive material comprises a cross-linked, derivatized atelocollagen that has been cross-linked in the presence of at least two different functionalized crosslinking agents and a sequestered curing agent, where at least one of the two different functionalized crosslinking agents is a functionalized polyethylene glycol cros slinking agent.

In another aspect, an adhesive composition comprises a derivatized, atelocollagen present at a concentration of at least 35 mg/mL in combination with a sequestered curing agent.

In an additional aspect, an adhesive material composition comprises a derivatized, atelocollagen with a number average molecular weight of about 300 kDa, the derivatized, atelocollagen promotes adhesion to a biological tissue when crosslinked and cured.

In another aspect, an adhesive composition comprises a collagen solution or suspension comprising a derivatized atelocollagen comprising a number average molecular weight of about 300 kDa, a first crosslinking agent, a second crosslinking agent different from the first crosslinking agent, and a sequestered curing agent, in which the collagen, the first crosslinking agent, the second crosslinking agent and the sequestered curing agent react provide the adhesive composition when mixed together.

In an additional aspect, a pre-cured adhesive composition comprises a mixture of a collagen solution or suspension comprising a derivatized atelocollagen comprising a number average molecular weight of about 300 kDa, in which the collagen solution is present in a first vessel, a first crosslinking agent, a second crosslinking agent different from the first crosslinking agent, a sequestered curing agent, in which the first crosslinking agent, the second crosslinking agent and the sequestered curing agent are present in a second vessel separate from the first vessel, in which the collagen, the first crosslinking agent, the second crosslinking agent and the sequestered curing agent to provide an adhesive material when mixed together.

In another aspect, a pre-cured adhesive composition comprises a mixture of a collagen solution comprising a derivatized atelocollagen with at least about 75% of carboxyl sites of the atelocollagen being derivatized, a first crosslinking agent, a second crosslinking agent different from the first crosslinking agent, a sequestered curing agent, in which the collagen, the first crosslinking agent, the second crosslinking agent and the sequestered curing agent provide an adhesive material when mixed together.

In an additional aspect, a pre-cured adhesive composition comprises a mixture of a collagen solution comprising a derivatized atelocollagen with at least about 75% of carboxyl sites of the atelocollagen being derivatized, in which the derivatized collagen comprises a number average molecular weight of about 300 kDa or less, a first crosslinking agent, a second crosslinking agent different from the first crosslinking agent, and a sequestered curing agent, in which the collagen, the first crosslinking agent, the second crosslinking agent and the sequestered curing agent provide an adhesive material when mixed together.

In another aspect, a kit comprises derivatized atelocollagen, a first crosslinking agent, a second crosslinking agent different from the first crosslinking agent, a sequestered curing agent; and instructions for using the derivatized atelocollagen, the first crosslinking agent, the second crosslinking agent and the sequestered curing agent to provide an adhesive material.

In an additional aspect, a kit comprises collagen comprises an atelocollagen, a derivatization agent, a first crosslinking agent, a second crosslinking agent different from the first crosslinking agent, a sequestered curing agent, instructions for using the atelocollagen and derivatization agent to provide a derivatized atelocollagen, and instructions for using the provided derivatized atelocollagen, the first crosslinking agent, the second crosslinking agent and the curing agent to provide an adhesive material.

In another aspect, a method of repairing a tissue defect comprises disposing a composition at a defect site of the tissue, the composition comprising a derivatized atelocollagen comprising a number average molecular weight of less than or equal to 300 kDa, a first crosslinking agent, a second crosslinking agent different than the first crosslinking agent and a sequestered curing agent, and permitting the curing agent to remain on a surface of the tissue for a curing period.

In an additional aspect, a method of repairing an epithelial tissue defect comprises disposing a composition at a defect site of the epithelial tissue, the composition comprising a derivatized atelocollagen comprising a number average molecular weight of less than or equal to 300 kDa, a first crosslinking agent, a second crosslinking agent different than the first crosslinking agent and a sequestered curing agent, and permitting the disposed composition to remain on a surface of the defect site for a curing period.

In another aspect, a method of repairing a tissue defect comprises disposing an adhesive composition at a defect site of the epithelial tissue, the adhesive composition comprising a derivatized atelocollagen, a first crosslinking agent, a second crosslinking agent different than the first crosslinking agent and an encapsulated curing agent, and permitting the composition to remain on a surface of the defect site for a curing period.

In an additional aspect, a method of repairing an epithelial tissue defect comprises disposing an adhesive composition at a defect site of the epithelial tissue, the adhesive composition comprising an alkylated atelocollagen, a first crosslinking agent, a second crosslinking agent different than the first crosslinking agent and a sequestered curing agent, and permitting the composition to remain on a surface of the defect site for a curing period.

In another aspect, a kit comprises collagen comprising an atelocollagen, a derivatization agent, a first crosslinking agent, a second crosslinking agent different from the first crosslinking agent, a first curing agent component, a second curing agent component, instructions for using the atelocollagen and derivatization agent to provide a derivatized atelocollagen, instructions for using the first curing agent component and the second curing agent component to provide a sequestered curing agent, and instructions for using the provided derivatized atelocollagen, the first crosslinking agent, the second crosslinking agent and the provided sequestered curing agent to provide an adhesive material.

In an additional aspect, an adhesive material comprises a cross-linked, derivatized atelocollagen that has been cross-linked in the presence of at least two different functionalized crosslinking agents and a sequestered curing agent.

In another aspect, a curing system for curing a bioadhesive comprises a sequestering agent and a curing agent.

In an additional aspect, a method comprises curing an adhesive disposed on a tissue by applying an effective amount of a sequestered curing agent to the disposed adhesive.

In another aspect, a method of treating bone damage comprises disposing the adhesive material described herein on a first bone surface to hold the first bone surface to a bone piece without the use of any mechanical fasteners.

In another aspect, a method comprising subjecting dry atelocollagen particles to an acidic solution comprising a derivatizing agent to provide the derivatized collagen, washing the derivatized collagen, and drying the derivatized collagen is provided. In certain instances, the acidic solution comprising the derivatizing agent comprises methanol in hydrochloric acid. In other examples, the atelocollagen particles comprise a mixture of mammalian, predominately Type I, either dermis or tendon-derived, predominantly monomeric atelocollagen (e.g., more than 50% by weight) in dry form. In some examples, the washing step comprises washing the derivatized collagen with an alcohol. In further embodiments, the alcohol is a primary alcohol. In some instances, the primary alcohol is selected from the group consisting of methanol, ethanol and propanol. In other examples, the drying step comprises drying using a vacuum. In some instances, the method comprises adding the dried, derivatized collagen to an acidic solution for storage. In other instances, the method comprises selecting the concentration of the derivatized collagen to provide a final concentration of about 50-70 mg/mL of the derivatized collagen. In further examples, the method comprises disposing the derivatized collagen onto a tissue site and curing the disposed derivatized collagen using a sequestered curing agent. In some instances, the curing step comprises disposing a sequestered sodium or calcium curing agent on the disposed derivatized collagen. In some examples, the sequestered sodium or calcium curing agent is disposed on the disposed derivatized collagen as a solid.

Other aspects and advantages of the present technology will become apparent to those skilled in the art, given the benefit of this summary and the following detailed description.

### BRIEF DESCRIPTION OF FIGURES

Certain configurations are described with reference to the accompanying figures in which:

FIG. 1A is an illustration of a NHS-PEG crosslinking agent; and

FIG. 1B is an illustration of a SH-PEG crosslinking agent.

### DETAILED DESCRIPTION

Certain embodiments are described below that are directed to collagen based adhesives that can be used in tissue repair procedures to hold or approximate two or more tissues or anatomical structures to each other. While the exact tissue repair method can vary, in a typical procedure the adhesive is disposed on one or both of two or more tissues and the tissues are contacted to each other for a period that permits the adhesive to cure and hold the tissues to each other. In some instances, the adhesive material can be smeared, smoothed, etc. to spread the material as a thin layer over the tissues. Once spread to a desired thickness, the material may then be cured by the addition of a suitable sequestered curing agent. In other instances, the collagen can be mixed with the sequestered curing agent prior to disposal on one or more tissues and the resulting combined material can be permitted to cure to hold the tissues to each other. If desired, the tissues can also be sutured, stapled or tissues can be coupled to each other through other mechanical means, e.g., screws, fasteners, etc. in addition to disposal of the adhesive material on the tissue(s). In other instances, the adhesive material may be the sole material which holds the tissues together and no external mechanical fasteners or devices may be present or used with the adhesive material.

While the exact thickness of the cured adhesive material can vary, a typical use thickness may vary from about 0.1 mm to about 2 mm, more particularly about 0.25 mm to about 1.5 mm, for example about 0.5 mm to about 1 mm. In some instances, it may be desirable to apply a thinner layer of material, cure it, and then apply additional material (and cure it) on top of the cured material. The number of layers applied may vary from about two to about ten or more. In certain configurations, layering of the material may be performed after an underlying layer is partially cured but not yet fully cured. In other instances, different materials can be layered or the same material can be layered but different sequestered curing agents (or sequestered curing agents of the same material but with a different average particle sizes) can then be added over a cured or partially cured layer.

The adhesive materials can be designed to be biocompatible, e.g., tissue compatible, and may be non-immunogenic and/or non-allergenic to permit wide uses in many different tissues. Further, the adhesive material can be cured without any substantial generation of heat which could cause tissue damage.

Curing Agents, Sequestration Agents and Curing Conditions

In certain embodiments, the curing agents described herein may be sequestered in that they are present in encapsulates, agglomerates, coacervates, microparticles, nanoparticles, liposomes or other structures which can act to bind to or associate with, e.g., electrostatically, through hydrogen bonding, through van der Waals interactions, etc., the curing agent. For example, the curing agent may be combined with one or more other materials to promote formation of higher molecular weight structures that can act to tie up or sequester at least some of the curing agent for at least some period. By sequestering some of the curing agent for some period, the slow use and/or release of the curing agent from the sequestered complexes can act to increase the overall adhesive strength of the cured adhesive compared to using the same curing agent without any sequestration agents.

In certain embodiments, the sequestering agent may be selected to encapsulate the curing agent for at least some period. For example, one or more suitable sequestering agents may be mixed with the curing agent components to provide encapsulated curing agents. While the exact type and amount of sequestering agent used may vary, illustrative sequestering agents including, but are not limited to, an alginate, a polysaccharide, a glycosoaminoglycan, a gum, a polyalcohol, liposomes, phospholiposomes, and other materials which can act to slow the exposure of the derivatized collagen materials to the curing agent. In some examples, the sequestering agent may comprise a molecular weight of at least 10 kiloDaltons, more particularly a molecular weight of at least 100 kiloDaltons, for example, a molecular weight of at least 200 kiloDaltons. The sequestering agent may be a naturally occurring material or may be a synthetic material or may comprise both a naturally occurring material and a synthetic material. As noted herein, the sequestered curing agent may comprise a complex of curing agent and sequestering agent.

In some instances, the sequestered curing agent may be produced by forming microparticles produced from an alginate, e.g., sodium alginate, in combination with one or more curing agents such as calcium salts. For example, sodium alginate can be used as an encapsulating agent due to its relative inexpensive cost and simplicity in producing microparticles. A similar microparticle may be produced using poly(vinyl alcohol) or other unsaturated polyols. In other instances, hyaluronic acid may be used to encapsulate the curing agent including both low molecular weight (MW) hyaluronic acid (MW<500 kDa) and/or high molecular weight hyaluronic acid (MW>500 kDa).

In some instances, sequestered curing agent can be formed as the curing agent ions displace the ions of the sequestering agent. For example, many of the sequestering agent may be present in sodium or potassium form, and the addition of a calcium based curing agent to the sequestering agent can result in displacement of the sodium or potassium by the calcium with sequestration of the calcium ions. For example, sodium ions in a sodium alginate molecule can be displaced by divalent ions, such as calcium, forming a higher ordered complex with adjacent alginate chains. In some instances, monovalent sodium ions in sodium alginate (or other agents) can be displaced by divalent calcium (or other divalent ions). Calcium can bind to two neighboring alginate chains instead of sodium, which can only bind to one. Specifically, divalent ions can bind to or associate with guluronate groups located on alginate. Given the divalent nature of the ion, guluronate groups on one alginate chain are able to couple with guluronate groups on a second alginate chain which brings the chain in proximity to each other. While calcium ions are described by way of example, other divalent curing ions such as magnesium or even certain transition metal divalent ions can instead be used as curing agents. In alternative embodiments, trivalent ions such as Al⁺³, Fe⁺³ or other trivalent metal ions can be used as or with curing ions. The exact curing agent selected may depend, for example, on the solubility of the materials in aqueous media and/or the desired concentration of curing agent to be used.

In other instances, the curing agent and sequestering agent may form a coacervate. Coacervates generally refer to a liquid phase separation caused by the association of oppositely charged polyions. Without wishing to be bound by any particular theory, derivatized collagen, e.g., alkylated collagen, which possesses an abundance of positive charge due to protonation of amine groups, and alginate, which is anionic (and in the presence of sodium carbonate, a weak base) could associate into a supramolecular complex (a coacervate) of both polymers (mostly driven by enthalpy). The presence of a complex coacervate is consistent with a noticeable drop in viscosity that is observed when the sequestered curing agent is mixed with the derivatized, cross-linked collagen, as noted in more detail below. These coacervates can be desirable for the use in a tissue adhesive as they function well in wet environments (such as moist tissue). Furthermore, use of hyaluronic acid instead of sodium alginate may produce a similar complex coacervate since hyaluronic acid is also anionic and is structurally similar to sodium alginate. In addition, the drop in viscosity by using a sequestered curing agent, e.g., as a result of forming a coacervate or other complex structure, can permit higher loadings/concentrations of collagen to be used in the adhesive, which can further increase overall adhesive strength.

In some examples, the sequestered curing agents may be produced and used in particle form as solids. While the exact particle size may vary depending on the materials used, the particle size is not limited and desirably the particles are large enough so unreacted sequestering agent can be separated from the adhesive material post-crosslinking. In addition, if the particles are too large, they have the potential to create voids or gaps in the adhesive layer, which could drastically reduce the adhesive strength, producing a weaker bond between two tissue planes. While not wishing to be bound by any particular theory, an informal upper limit can be approximated that is less than the thickness of the applied adhesive layer. For example, the microparticles desirably have an average particle diameter that is less than the thickness of adhesive deposited on a tissue surface. If the average particle diameter exceeds the thickness of the adhesive layer, unwanted “peaks” in the adhesive layer may be produced that interfere with the surface contact of the two tissue layers, which could limit the adhesive strength. If it is desired to minimize the actual thickness of the adhesive layer, then an upper adhesive layer thickness of about 0.5 mm can be used as a guide. If the adhesive layer is too thick, the adhesive layer may actually act as a barrier and inhibit the adhesion of the two tissue planes. Also, if the adhesive layer is too thick, a longer cure time than desired may be required. The exact curing agent particle size can be selected by taking into account the above considerations.

In certain embodiments, to prepare the sequestered curing agents, each of the curing agent and the sequestering agent can be dissolved in a suitable solvent. In some instances, the solvent can be water, a buffer solution or an organic solvent with a relatively high dielectric strength, e.g., acetone, methanol or ethanol optionally mixed with water. In certain configurations, the solvents used for dissolving the components may be the same, whereas in other instances the solvents may be different. In some examples, each solution may be co-sprayed into a container where the particles may be permitted to form. In other instances, the solutions can be mixed, and the resulting mixture can be sprayed into a container to permit the particles to form. In some configurations, one of the solutions may be present in a container, and the other solution can be sprayed into (or otherwise introduced) into the solution. For example, one of the solutions may be air sprayed into the other solution with mixing to permit the particles to form. In additional instances, the solutions can be mixed and the resulting mixture may be lyophilized to form the particles. Once the particles of sequestered curing agent are formed, the particles can be washed, centrifuged, sized and/or processed in some manner to remove the sequestered curing agent from non-sequestered curing agent and/or free sequestering agent. The particles may then be dried to remove any or substantially all moisture from the particles, e.g., may be air-dried, dried in an oven, dried under vacuum or dried using other means. Centrifugation, filtering, and other operations may also be performed to separate the sequestered curing agent from any liquids. In other instances, the particles may remain wet or damp prior to addition to the other components of the adhesive material.

In some embodiments, the sequestered curing agents may not include any aldehyde compounds, e.g., glutaraldehyde, as such compounds can often result in killing of cells. In particular, glutaraldehyde free compositions comprising cured, derivatized and cross-linked atelocollagen materials are particularly desirable for use in applications that contact living cells or tissues. As noted herein, crosslinking of the collagen can result in eventual formation of an adhesive or gel which may be deposited onto a desired tissue site.

Illustrative curing agents that can be mixed with one or more sequestering agents to form a sequestered curing agent include but are not limited to sodium salts (e.g., sodium chloride, sodium nitrate, sodium phosphate, sodium carbonate, sodium acetate, sodium citrate, sodium bicarbonate, sodium bisulfate, sodium perchlorate, sodium gluconate, sodium lactate), potassium salts (e.g., potassium chloride, potassium nitrate, potassium phosphate, potassium carbonate, potassium acetate, potassium citrate, potassium bicarbonate, potassium bisulfate, potassium perchlorate, potassium gluconate, potassium lactate), calcium salts (e.g., calcium chloride, calcium carbonate, calcium phosphate, calcium acetate, calcium citrate, calcium gluconate, calcium lactate) and mixtures thereof. Any one or more of the salts can be dissolved in a buffer or solvent at a desired pH, e.g., a pH of less than 7, a pH of about 7 to about 7.5 or a pH greater than 7.5. For example, where a basic curing agent solution is desired, a pH of about 8-8.5 may be used. Where an acidic curing agent solution is desired, a pH of about 6-6.5 may be used. In some instances, the one or more salts can be added directly to water without any pH adjustment. Once the salts are dissolved, the curing agent solution may be combined with one or more sequestering agents as described herein to provide a sequestered curing agent. The sequestered curing agent can be mixed with the crosslinking agents or may be added to the collagen material concurrently with the crosslinking agents or after the crosslinking agents have been mixed with the collagen.

In other instances, the salt or salts used as curing agents may comprise divalent cations. For example, calcium or magnesium salts can be used either alone or together. In other instances, the divalent cation may be a divalent transition metal, e.g., copper, silver, cobalt, etc. The exact anion used with the divalent cation salt may vary and includes, but is not limited to, halides, carbonates, nitrates, sulfates and other anions. In some examples, the anion may be selected such that the divalent cation salt is minimally water soluble at room temperature.

In some embodiments, desired physical properties of the collagen material can be achieved by using sequestered solid or sequestered powder curing agents, e.g., sequestered curing agents lacking any liquid and/not dissolved in any solvent. Strength may be enhanced by using sequestered, solid or sequestered, powder curing agents which can act to dehydrate the gel and increase the overall collagen weight percentage (and reduce the percentage by weight of water) of the resulting deposited material. In some embodiments, the sequestered curing agent can be sprayed on using an air brush or other desired devices. In certain instances, a first sequestered curing agent is deposited or blown onto the surface of a deposited collagen material, and then an optional second sequestered curing agent may be deposited or blown onto the first curing agent. Alternatively, a first sequestered curing agent can be mixed with the collagen and the crosslinking agent and a second curing agent (either a sequestered curing agent or a non-sequestered curing agent) can then be added to the mixture. This two-step curing may improve properties relative to a single step curing. In other instances, however, two or more sequestered curing agents may first be mixed and then added or blown onto the deposited collagen gel to permit curing to occur (or can be simultaneously mixed the collagen and the crosslinking agents to permit curing to occur). Where sequestered solid curing agents are used, the agents may be any of those curing agents described herein in connection with agents that can be dissolved or are used in a liquid form. In some instances, a first curing agent present as a liquid can be deposited on the collagen, and a second sequestered curing agent present as a solid can be added or blown into the first curing agent. If desired, a first sequestered curing agent present as a solid can be added or blown onto the deposited collagen, and a second curing agent present as a liquid (which may be sequestered or non-sequestered) can deposited on the first curing agent. In additional configurations, a first solid, sequestered curing agent can be mixed with the collagen and crosslinking agents and additional curing agent (in solid or liquid form) can be added to the mixture later.

In certain embodiments, excess curing agent may be added to ensure full curing takes place in a desired time, e.g., 10 minutes, 15 minutes, 20 minutes or any value between these values such as 10-15 minutes. If desired, any excess curing agent can be removed by wiping, washing or otherwise adding some material to the surface of the deposited collagen to remove any residual curing agent. In other instances, excess curing agent may be permitted to remain in place without any washing steps or wiping steps. In some instances, excess sequestered curing agent may remain in solid form after the adhesive material is formed. The excess sequestered curing agent may be washed or rinsed away using water, saline or other liquids. In some instances, an amount of curing agent used is selected such that substantially all curing agent is consumed or used during the curing process. For example, the amount of curing agent added may be about 50% by weight of the amount of adhesive component (by weight) used. In other instances, the curing agent may be added in excess to ensure full cure of the adhesive takes place.

In certain embodiments, a film of sequestered curing agent may be placed over the disposed, crosslinked collagen or atelocollagen. For example, a film may be cast from a solution comprising one or more sequestered curing agents optionally in combination with one or more carriers such as, for example, a polysaccharide, a glucan, a dextran or other suitable carriers. The presence of the sequestering agent may permit formation of a film without the use of any carriers. In use, the solid film of curing agent may be disposed onto an area comprising spread crosslinked, derivatized collagen or atelocollagen and may remain in contact with the crosslinked, derivatized collagen or atelocollagen for a curing period. In some instances, the sequestered curing agent film can then be removed, whereas in other instances, the sequestered curing agent film may remain in place. Illustrative materials which can be used to provide a sequestered curing agent film include, but are not limited to, sodium salts (e.g., sodium chloride, sodium nitrate, sodium phosphate, sodium carbonate, sodium acetate, sodium citrate, sodium bicarbonate, sodium bisulfate, sodium perchlorate, sodium gluconate, sodium lactate), potassium salts (e.g., potassium chloride, potassium nitrate, potassium phosphate, potassium carbonate, potassium acetate, potassium citrate, potassium bicarbonate, potassium bisulfate, potassium perchlorate, potassium gluconate, potassium lactate), calcium salts (e.g., calcium chloride, calcium carbonate, calcium phosphate, calcium acetate, calcium citrate, calcium gluconate, calcium lactate) and mixtures thereof.

In some embodiments, the film can be used to spread the derivatized, crosslinked atelocollagen, and the film may comprise sufficient materials to permit spreading without substantial tearing of the film. The film may also permit the salts present in the curing agent film to dissolve on contact with the derivatized, crosslinked atelocollagen. In some instances, one or more plasticizers can be added to the film to alter the properties of the curing agent film.

In certain examples, one or more additional stimuli such as cooling, light, chemical initiators or other physical or chemical stimulus can also be added or used to assist in curing of the derivatized, crosslinked atelocollagen. For example, the salt solutions used in certain curing agents may act to cure the derivatized, crosslinked atelocollagen from the outside in with the outer portion curing faster than a portion adjacent to the tissue. To assist in curing underlying material of the derivatized, crosslinked atelocollagen, one or more stimuli may be applied.

In certain instances, the sequestered curing film itself may be used to spread the crosslinked, derivatized collagen or atelocollagen at a defect site. For example, in use of the crosslinked, derivatized collagen or atelocollagen, the material is typically dispensed from a syringe over a tissue repair site, e.g., a site where two tissues are to be held together. The material may then be spread to a thin film of a desired thickness, e.g., about 0.1 mm to about 1.5 mm, using a suitable tool such as a thin rigid piece of plastic or other materials. If desired, the sequestered curing agent film can be cast onto the surface of the plastic tool such that spreading of the crosslinked, derivatized collagen or atelocollagen results in contact of the crosslinked, derivatized collagen or atelocollagen with the sequestered curing agent film. In other instances, the sequestered curing agent film itself may be rigid enough to spread the material over the defect site and at the same time cure the crosslinked, derivatized collagen or atelocollagen as the sequestered curing agent film spreader contacts the crosslinked, derivatized collagen or atelocollagen.

In certain embodiments, additional materials, additives, etc. may also be used with the collagen and/or curing agent. For example, isoprene based materials may be added to the curing agent and/or derivatized collagen to alter the flexibility of the cured material. In other instances, a biological material such as, for example, elastin may be included to mimic tissue flexibility. In further instances, powders, whiskers, fibers, particles, nanoparticles or other materials may also be included in the sequestered curing agent or mixed with the derivatized collagen material.

In some embodiments, the sequestered curing agents used herein may permit formation of an adhesive material that is adherent for a sufficient period to permit the tissue to repair themselves but is not necessarily designed to act as a permanent adhesive. For example, the sequestered curing agent and other materials can be selected to provide a bioadhesive that serves as a temporary interface while the body's natural healing mechanisms repair the tissue damage and reestablish the bonding of two separated tissue planes. The separation of tissue layers is commonly encountered in medicine. The development of seromas, which is an accumulation of fluid between tissue layers, is a critical problem and one example of a possible use for the adhesives described herein is to avoid formation of seromas. In some instances, the adhesive may provide adherence between the tissues in situ for at least 7 days, more particularly, at least 10 days or at least 14 days. In certain embodiments, the adhesive strength of the adhesive materials may be at least 50 N as tested by ASTM F2255 dated 2003, more particularly at least 60 N as tested by ASTM F2255 dated 2003 or at least 70 N as tested by ASTM F2255 dated 2003. In certain instances, the T-peel strength as tested by ASTM F2256 dated 2005 may be at least 0.20 N, for example, at least 0.50 N as tested by ASTM F2256 dated 2005 or at least 0.70 N as tested by ASTM F2256 dated 2005. During the tissue repair process, the bioadhesive may be resorbed, degraded, etc. or may be used as a makeshift framework to permit cell ingrowth and/or stabilization during the tissue repair process.

In use of the sequestered curing agents, the mixture comprising the various components is typically permitted to cure for a curing period while the tissues are held together. For example, the tissues may be held together using forceps or other mechanical means until the adhesive has had sufficient time to cure to hold the tissues together without the use of the forceps or other means. Cure temperature typically is room temperature or above, e.g., 25° C. or 37° C., and slightly elevated temperatures above human body temperature, e.g., 38-40° C., may be used for certain period to assist in the curing process.

Collagens and Atelocollagens

In certain embodiments, the compositions described herein may comprise one or more collagens or atelocollagens. An atelocollagen can be produced from a collagen by digesting the collagen with a protease, e.g., pepsin, or other suitable enzymes to remove the atelopeptides from the collagen helix. Without wishing to be bound by any theory, collagen comprise a triple helix including a length of about 300 nm and a weight (in monomeric triple helix form) of about 300 kiloDaltons (kDa). In certain examples, the particular form of collagen selected for use in producing the materials described herein can vary depending on the desired end product and/or the desired types of collagen structures present from different tissues. In some embodiments, collagen obtained from any source, including both naturally occurring collagens and synthetic collagens, can be used to provide the materials described herein. For example, collagen may be extracted and purified from human or other animal sources, e.g., mammalian sources, such as bovine or porcine, or may be recombinantly produced, synthetically produced by chemical synthesis or otherwise produced using selected techniques. Collagen of any type, including, but not limited to, types I-IV or any combination thereof, may be used. In some embodiments, type I collagen is used to provide the materials described herein. In other embodiments, atelopeptide collagen or telopeptide-containing collagen may be used. Atelopeptide collagen, a.k.a., atelocollagen, may include desirable attributes including reduced immunogenicity when compared to telopeptide-containing collagen. In some embodiments, the collagen can be a Type I collagen, a Type II collagen, a Type III collagen, a Type IV collagen, a gelatin, a collagen comprising agarose, a collagen comprising hyaluronan or hyaluronic acid, a collagen comprising proteoglycan, a glycosaminoglycan, a glycoprotein, glucosamine or galactosamine, a collagen comprising fibronectin, a collagen comprising laminin, a collagen comprising a bioactive peptide growth factor, a collagen comprising a cytokine, a collagen comprising elastin, a collagen comprising fibrin, a collagen comprising polylactic, polyglycolic or polyamino acid, a collagen comprising polycaprolactone, collagen comprising a polypeptide, or a copolymer thereof, each alone or in combination. In other examples, the collagen can be prepared from, or can include, collagen precursors, such as, for example, peptide monomers, alpha 1 (type I), and alpha 2 (type I) collagen peptide or alpha 1 (type I) alpha 2 (type I) peptides, alone or in combination, or from a combination of precursors, such as 2 (alpha 1, type I) peptide and 1 (alpha 2, type I) peptide. If desired, the collagens can be used with other compounds, such as pharmaceutically acceptable excipients, surfactants, buffers, additives and other biocompatible components or pharmaceutical agents, therapeutic agents or bioactive agents or molecules.

In some examples, collagen can be isolated from animal tissues, e.g., skin, hides, etc. For example, the tissue can be digested in the presence of one or more enzymes to solubilize the collagen and provide an atelocollagen. Atelocollagens are typically lower in antigenicity than native collagen but can still provide many of the properties of an intact collagen. Atelocollagens can also be soluble (under certain conditions) which can further enhance processing and use. As described herein, however, certain instances can use a solid (and optionally suspended in a solution) atelocollagen which can be derivatized and then reacted with one or more crosslinking agents and/or sequestered curing agents to provide an adhesive material. In some instances, the solid collagen can be reacted with solid (powdered) crosslinking agent and solid (powdered) sequestered curing agents. These reactions and curing may take place in one or more vessels or containers, e.g. syringes, tubes, vials, etc. or may take place at a tissue site.

In some embodiments, the atelocollagen can be isolated and stored at temperatures below room temperature, e.g., less than 25° C., 15° C., 10° C., 4° C. or 0° C., to reduce the likelihood of formation of higher ordered structures. For example, collagen and atelocollagens can form triple helix dimers, triple helix trimers, etc. in solution. Formation of these higher ordered structures can reduce the level of monomeric atelocollagen in solution and may provide adhesives with less desired properties. In certain examples described herein, monomeric triple helix derivatized atelocollagen can be present in a major amount by weight, e.g., more than 50% by weight, in the collagen. In some instances, the collagen may comprise derivatized atelocollagen comprising a number average molecular weight of 300 kDa or less, e.g., a major amount of derivatized atelocollagen comprising a number average molecular weight of 300 kDa or less may be present in the collagen used to provide the adhesive or other material. Without wishing to be bound by any particular theory, where derivatized atelocollagen comprising a number average molecular weight (NAMW) of 300 kDa or less is used, the overall viscosity (per mg/mL of the material) is reduced compared to collagens used in other collagen based sealants and adhesives. By having a lower viscosity, increased amounts of the derivatized atelocollagen can be present for a particular viscosity, e.g., 40 mg/mL derivatized atelocollagen with a NAMW of 300 kDa can have about the same viscosity as 20 mg/mL of collagen used for prior collagen based adhesives such as those described in U.S. Pat. No. 6,458,889, for example. In addition, the use of the sequestered curing agents can lower the viscosity further, which can permit higher loading of collagen, e.g., 45 mg/mL, 50 mg/mL or higher of derivatized atelocollagen with a NAMW of 300 kDa, when the collagen is used in combination with a sequestered curing agent. By having a higher concentration of derivatized atelocollagen present, improved properties can be achieved while maintaining a suitable viscosity for use as an adhesive. It is desirable for the viscosity to be high enough that the adhesive does not “run off” the surface and remains localized to the selected area of application. The viscosity should also be low enough to possess fluidity to fill in defects and imperfections present in the tissue surface, enabling better contact and adhesion.

In certain embodiments, an atelocollagen from porcine sources can be isolated and purified and stored at a temperature of room temperature or below prior to use. The porcine atelocollagen can be derivatized, e.g., alkylated, under suitable conditions, e.g., in the presence of an alkylating agent and/or acid. Alkylation may result in a solid derivatized collagen that can then be used to provide an adhesive, gel or similar material. In some instances, the porcine, derivatized atelocollagen can then be reacted with one, two, three or more crosslinking agents (as described below) to provide an adhesive or similar material. The porcine, derivatized, cross-linked atelocollagen may then be cured using suitable curing agents, e.g., liquid or solid curing agents, to provide a material with desirable properties.

Derivatization Methods and Reagents

In certain embodiments, the collagen (or atelocollagen) can be derivatized by reacting the collagen with a suitable agent that can form a covalent bond with one or more groups present in the collagen structure. The exact derivatization conditions may vary and may be performed in liquid solution or using gaseous reagents to react with the collagen (or atelocollagen). In some instances, the derivatization process may be performed in a single step in a single reaction vessel, whereas in other instances the derivatization process may be performed in two or more steps and optionally in two or more different vessels.

In some embodiments, the collagen can be derivatized using a suitable alkyl alcohol in the presence of an acid or base (depending on the alcohol used and the desired reaction to be promoted). Illustrative alkyl alcohols include, but are not limited to, methanol, ethanol, propanol, isopropanol, butyl alcohol, isobutyl alcohol, alcohols with alkyl chains having five to eight carbons (saturated or unsaturated), alcohols with alkyl chains having nine to twelve carbons (saturated or unsaturated), alcohols with alkyl chains having thirteen to eighteen carbons (saturated or unsaturated), alcohols with alkyl chains having nineteen to twenty-four carbons (saturated or unsaturated) and substituted forms of these illustrative alcohols. In some embodiments, the derivatizing agent may comprise one or more free hydroxyl groups (-OH) but may not be considered an alcohol, e.g., may be considered a carboxylic acid, an epoxide, an aldehyde or other groups.

In some examples, the collagen or atelocollagen can be derivatized by reacting one or more collagens with a suitable derivatizing agent which may be a pharmaceutically active compound or a pharmaceutically inactive compound. In some embodiments, the derivatizing agent includes at least one carbon atom and a suitable reactive group that can react with one or more groups present in the collagen or atelocollagen. For example, the derivatizing agent can include a hydroxyl group, an amino group or both that can react with a suitable group present on the collagen to couple the derivatizing agent to the collagen. In some embodiments, the derivatizing agent can take the form of an alkylating agent, e.g., an agent that can add a methyl group, ethyl group, propyl group or other alkyl group to the collagen, e.g., C1-C6 alkyl groups that may be saturated or unsaturated or C6-C24 alkyl groups which may be saturated or unsaturated. Alkylated atelocollagens may be particularly desirable due to their potential for improved solubility in a variety of common solvents, the potential for more facile formulation with drugs and various other small-molecule and/or macromolecular additives, and the potential for good mechanical properties resulting from intermolecular interactions between alkyl groups, especially alkyl groups of sufficient length to promote hydrophobic association in aqueous solutions and even crystallization-induced aggregation. Also, the adhesive properties of such modified atelocollagens can be tuned by judicious choice of alkyl-based functional groups, e.g., alkyl groups including long hydrocarbon chains, and spacing of the functional groups along the collagen polymer chain.

In some embodiments, more than a single type of derivatizing agent can be used. For example, collagen or atelocollagen can first be reacted with a first derivatizing agent and then subsequently reacted with a second derivatizing agent. In other embodiments, the same derivatizing agent can be used, but it may be reacted sequentially with the collagen molecule or reacted in selected stoichiometric amounts with the collagen to derivatize a desired proportion of the collagen. In some embodiments, the collagen may first be derivatized with a lower molecular weight alkyl chain (C1-C6) and then subsequently reacted with a higher molecular weight alkyl chain (C10-C18 or C10-C24) to increase the overall hydrophobicity of the derivatized collagen. In other embodiments, the collagen can first be reacted with a lower molecular weight alkyl chain and subsequently reacted with a different group, e.g., a pharmaceutically active compound or a biological agent such as, for example, a peptide, protein, carbohydrate, lipid or other biological agents.

In certain embodiments, the derivatizing agent can be an alkylating agent having one to six carbon atoms including, but not limited to, straight chain, branched and cyclic alkylating agents with one to six carbon atoms. Where 5 or 6 carbon atoms are present, the alkylating agent can be a fully saturated cyclic compound, a cyclic compound with one or more sites of unsaturation, or an aromatic compound. In other embodiments, the alkylating agent can include one carbon, one to two carbons, one to three carbons, one to four carbons, one to five carbons, or one to six carbons. In some examples, the alkylating agent can include two carbons, two to three carbons, two to four carbons, two to five carbons or two to six carbons. In other examples, the alkylating agent can include three carbons, three to four carbons, three to five carbons, or three to six carbons. In certain examples, the alkylating agent can include four carbons, four to five carbons or four to six carbons. In some embodiments, the alkylating agent can include five carbons or five to six carbons. In other embodiments, the alkylating agent can include six carbons. The alkylating agents described herein may be fully saturated or may be present in unsaturated form, e.g., may include one or more double or triple bonds. In some instances herein, alkylating agents including one to six carbon atoms are referred to as “lower weight alkylating agents.”

In certain embodiments, the alkylating agent can include six to ten carbons. Alkylating agents with six to ten carbon atoms are sometimes referred to herein as “middle weight alkylating agents.” In certain examples, the alkylating agent can include six to ten carbons, six to nine carbons, six to eight carbons, or six to seven carbons. In other embodiments, the alkylating agent can include seven to ten carbons, seven to nine carbons or seven to eight carbons. In further embodiments, the alkylating agent can include eight to ten carbons or eight to nine carbons. In other examples, the alkylating agent can include nine carbon atoms or ten carbon atoms. The middle weight alkylating agents described herein may be fully saturated or may be present in unsaturated form, e.g., may include one or more double or triple bonds.

In other embodiments, the alkylating agent can be selected to include more than ten carbon atoms to provide a hydrophobic, derivatized collagen. Alkylating agents including more than ten carbon atoms are referred to herein in certain instances as “high weight alkylating agents.” In some instances, derivatized atelocollagens can be more hydrophobic than native collagen and can be adherent, at least to some degree, to permit use of the derivatized collagen in wound repair, defect repair, skin grafts, as scaffolds to attract cells and permit colonization by cells or other uses. The hydrophobic nature of the derivatized atelocollagen may result in the collagen precipitating out of solution and being considered a solid. The solid can be suspended if desired to permit further reaction. Due to the sticky nature of certain collagens after derivatizing with an alkylating agent, they can also be used as glues or adhesives, or when used as an implant or scaffold can be used without an additional adhesive. While not required, “high weight alkylating agents” may comprise up to eighteen, nineteen, twenty, twenty-one, twenty-two, twenty-three, or twenty-four carbons. In some instances, high weight alkylating agents can take the form of a fatty acid chains such as those found in essential fatty acids and non-essential fatty acids.

In certain examples, where the alkylating agent includes more than ten carbon atoms, it may include ten to twenty four carbons, ten to twenty-two carbons, ten to twenty carbons, ten to eighteen carbons, ten to sixteen carbons, ten to fourteen carbons or ten to twelve carbons and include both saturated and unsaturated forms thereof. In other embodiments, the alkylating agent can include twelve to twenty four carbons, twelve to twenty-two carbons, twelve to twenty carbons, twelve to eighteen carbons, twelve to sixteen carbons, or twelve to fourteen carbons and include both saturated and unsaturated forms thereof. In further embodiments, the alkylating agent can include fourteen to twenty four carbons, fourteen to twenty-two carbons, fourteen to twenty carbons, fourteen to eighteen carbons, or fourteen to sixteen carbons and include both saturated and unsaturated forms thereof. In additional embodiments, the alkylating agent can include sixteen to twenty four carbons, sixteen to twenty-two carbons, sixteen to twenty carbons, or sixteen to eighteen and include both saturated and unsaturated forms thereof. In other embodiments, the alkylating agent can include eighteen to twenty four carbons, eighteen to twenty-two carbons, or eighteen to twenty carbons and include both saturated and unsaturated forms thereof. In some embodiments, the alkylating agent can include twenty to twenty four carbons or, twenty to twenty-two carbons and include both saturated and unsaturated forms thereof. In embodiments where long alkyl chains, e.g., C18 or more, are to be added to the collagen or atelocollagen methods similar to those described by Greenberg and Alfrey in “Side Chain Crystallization of n-Alkyl Polymethacrylates and Polyacrylates,” J. Am. Chem. Soc., 1954, 76 (24), pp. 6280-6285, can be used.

In some embodiments, the alkylating agent comprises at least one terminal reactive group, e.g., a terminal hydroxyl group or terminal amino group. In other embodiments, the alkylating agent comprises at least one internal reactive group, e.g., a hydroxyl group or amino group not present at a terminus of the alkylating agent. In some embodiments, the alkylating agent can be a compound of formula (VI)

R₁−CH₃ (VI)

where R₁ may be hydroxyl, amino, acyl, or benzoyl. In other embodiments, R₁ includes at least one carbon atom, e.g., 1 to 6 carbon atoms, in addition to a non-carbon atom such as, for example, hydrogen, nitrogen or sulfur. In some embodiments, R₁ can be HOCH₂-, HOCH₂CH₂-or HO(CH₂)ₙ- where n is three to ten, more particularly, three to nine, three to eight, three to seven, three to six, three to five, or three, four, five, six, seven, eight, nine or ten. In some embodiments, R₁ of formula (1) is hydroxyl (-OH), a primary amine (-NH₂) or may be a diamine or a sulfhydryl or thiol group (-SH).

In other embodiments, the alkylating agent can be one or more of the compounds of formula (VII)

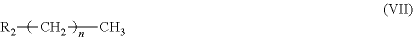

where R₂ may be hydroxyl, amino, acyl or benzoyl and n can be 1, 2, 3, 4, 5, or 6. In some embodiments, n is 1 and R₂ is hydroxyl, e.g., -OH. In other embodiments, n is 2 and R₂ is hydroxyl. In further embodiments, n is 3 and R₂ is hydroxyl. In additional embodiments, n is 4 and R₂ is hydroxyl. In additional embodiments, n is 5 and R₂ is hydroxyl. In some embodiments, n is 1 and R₂ is amino, e.g., -NH₂. In other embodiments, n is 2 and R₂ is amino. In further embodiments, n is 3 and R₂ is amino. In additional embodiments, n is 4 and R₂ is amino. In additional embodiments, n is 5 and R₂ is amino. If desired, where R₂ is amino, the amino group may be a secondary amine or a tertiary amine instead of a primary amine.

In certain embodiments, the alkylating agent can be selected as a compound of (VIII)

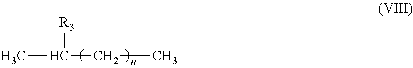

where R₃ may be hydroxyl, amino, acyl or benzoyl and n can be 1, 2, 3, 4, 5 or 6. In some embodiments, n is 1 and R₃ is hydroxyl, e.g., -OH. In other embodiments, n is 2 and R₃ is hydroxyl. In further embodiments, n is 3 and R₃ is hydroxyl. In additional embodiments, n is 4 and R₃ is hydroxyl. In additional embodiments, n is 5 and R₃ is hydroxyl. In some embodiments, n is 1 and R₃ is amino, e.g., -NH₂. In other embodiments, n is 2 and R₃ is amino. In further embodiments, n is 3 and R₃ is amino. In additional embodiments, n is 4 and R₃ is amino. In additional embodiments, n is 5 and R₃ is amino. If desired, where R₂ is amino, the amino group may be a secondary amine or a tertiary amine instead of a primary amine.

In some embodiments, the derivatizing agent can be a compound having formula (IX)

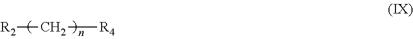

where R₂ of formula (IX) may be any of those groups listed for R₂ of formula (2) and n is 1, 2, 3, 4, 5 or 6. R₄ can methoxy, ethoxy, oxypropyl, hydroxyl, amino, acyl, benzoyl, aryl, naphthyl or carboxyl (-COOH). In some embodiments, each of R₂ and R₄ is hydroxyl and n is 2, 3, 4, 5, or 6. In other embodiments, one of R₂ and R₄ is hydroxyl and the other of R₂ and R₄ is amino and n is 2, 3, 4, 5 or 6.

In other embodiments, the derivatizing agent can be a compound having formula (X)

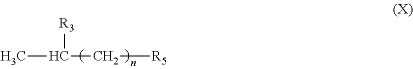

where R₃ of formula (5) may be any of those groups listed for R₃ of formula (3) and n is 1, 2, 3, 4, 5 or 6. R₅ can methoxy, ethoxy, oxypropyl, hydroxyl, amino, acyl, benzoyl, aryl, naphthyl or carboxyl. In some embodiments, each of R₃ and R₅ can be hydroxyl or one of R₃ and R₅ is hydroxyl and the other one of R₃ and R₅ is amino.

In other embodiments, the alkylating agent may be a compound of formula (XI)

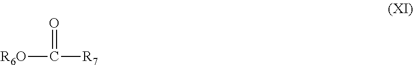

where R₆ can be hydrogen or a hydrocarbon chain (saturated or unsaturated) having 1 to 4 carbon atoms which may be branched if desired. In some embodiments, R₇ comprises 1 to 6 carbons (saturated and unsaturated forms). In other embodiments, R₇ may be a hydrocarbon chain (saturated or unsaturated) including 7-10 carbons. In further embodiments, R₇ may be a hydrocarbon chain (saturated or unsaturated) including ten to twenty four carbon atoms. In some embodiments, R₆ is hydrogen and R₇ is selected to provide compound (6) that is one of dodecanoic acid, tetradecanoic acid, hexadecanoic acid, octadecanoic acid, eicosanoic acid, tetracosanoic acid, 9-hexadecenoic acid, 9-octadecenoic acid, 9,12 octadecadienoic acid, 9,12,15-octadecatrienoic acid, 6,9,12-octadecatrienoic aid, 5,8,11,14-eicosatetraenoic acid or 15-tetracosenoic acid. In some embodiments, R₇ can have a formula of CH₃(CH)ₙ- where n can be nine to about twenty-three. In other embodiments, R₇ can have a formula of CH₃(CH)ₙ-C═O where n can be nine to about twenty-three. In some embodiments, R₆ is hydrogen and R₇ is any one of the groups listed above for R₇.

In other embodiments, the alkylating agent may be a compound of formula (XII)

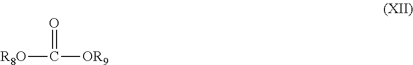

where R₈ can be hydrogen or a hydrocarbon chain (saturated or unsaturated) having 1 to 4 carbon atoms which may be branched if desired, and R₉ may be a hydrocarbon chain (saturated or unsaturated) including ten to twenty four carbon atoms. In some embodiments, R₉ can have a formula of CH₃(CH)ₙ- where n can be nine to about twenty-three. In other embodiments, R₉ can have a formula of CH₃(CH)n-C═O where n can be nine to about twenty-three. In some embodiments, each of R₈ and R₉ is hydrogen. In other embodiments, R₈ is hydrogen and R₉ may be any of the groups listed above for R₉.

In certain embodiments, the atelocollagen may be a methylated atelocollagen comprising one or more methyl groups added to the collagen at a suitable site. For example, the derivatized collagen may be methylated at one or more carboxyl groups to provide an ester. In some embodiments, enough derivatizing agent is present such that substantially all free and accessible carboxyl sites of the collagen react with the derivatizing agent to form an ester comprising a methoxy group covalently bonded to the carbonyl group of the ester.

In other embodiments, the derivatizing agent to provide methylation (or other alkylation or derivatization) can be selected such that free carboxyl groups remain in the collagen molecule subsequent to reaction with the derivatizing agent. By using selected amounts of the derivatizing agent, free reactive groups remain post-derivatization to permit reaction with other agents, e.g., one or more crosslinking agents.

In certain examples, the exact conditions used to derivatize the collagen or atelocollagen may depend on the particular derivatizing agent used. In some embodiments, derivatization may be acid catalyzed or base catalyzed. In additional instances, derivatization can be performed at room temperature, elevated temperature above room temperature or at a temperature below temperature. As noted herein, maintaining the collagen at reduced temperature may reduce the formation of unwanted higher order structures. The form of the derivatizing reagents used may also vary, e.g., gaseous forms and liquid forms can be used if desired.

In some embodiments, the collagen or atelocollagen may be derivatized in the presence of an alkyl alcohol, e.g., methanol, ethanol, propanol, etc. under acidic conditions. For example, gaseous or liquid acid may be present in a reaction vessel with the collagen or atelocollagen, and the alkyl alcohol can be introduced into the reaction vessel to derivatize the collagen or atelocollagen. The exact acid used may depend on the particular alkyl alcohol selected, but illustrative acids include but are not limited to acetic acid, citric acid, perchloric acid, oxalic acid, hydrochloric acid, hypochlorous acid, sulfuric acid, nitric acid, phosphoric acid, hypophosphorous acid, hypophosphoric acid and other acids which may or may not include metal species such as chromium, selenium, manganese or other metals. The pH of any solution used in the derivatization process is desirably not so low that the collagen or atelocollagen is degraded to any substantial degree, and illustrative pH values which can be used vary from about 2 pH units to about 6.5 pH units. If desired, one or more acidic buffers may also be present to keep the pH at a substantially constant pH during the derivatization process.

In certain embodiments, a pharmaceutically active compound can be reacted with the derivatized collagen or atelocollagen to conjugate the active compound to the collagen or atelocollagen. In some instances, the pharmaceutically active compound make take the form of a biological agent or compound, e.g., a peptide, protein, carbohydrate, lipid or the like, that can be conjugated to the derivatized collagens described herein. Illustrations of pharmaceutically active compounds are described in more detail below. In certain examples, a pharmaceutically active compound is a chemical compound, e.g., natural or synthetic, that can elicit a biological response, e.g., activate a cellular response or pathway, inhibit a cellular response or pathway, promote a cellular response or pathway, or alter a cellular response or pathway, under suitable conditions. For example, a pharmaceutically active compound can result in activation (or inactivation) of a cellular pathway, enzyme activation, enzyme inhibition, differentiation of a stem cell into a desired cell type, repair of a tissues, cellular enhancement, inhibition of cellular processes, activation (or deactivation) of an ion channel, an increase (or decrease) in protein expression, an increase (or decrease) in RNA production, an increase (or decrease) in mitosis, an increase (or decrease) in meiosis, an increase (or decrease) in intracellular transport, or other activities that a cell may perform or initiate through one or more cellular systems or pathways.

In certain examples, when a pharmaceutically active compound is present as a conjugate with a derivatized collagen or atelocollagen, e.g., a methylated atelocollagen, it may be present in an effective amount, e.g., an amount effective to elicit the biological response. In some configurations, the concentration of pharmaceutically active compound may exceed the amount desired to elicit a biological response such that sustained release of the pharmaceutically active compound can provide for such biological response for extended periods. It will be recognized by the person of ordinary skill in the art, given the benefit of this disclosure, that there are multiple ways to provide an effective amount of the pharmaceutically active compound. In some embodiments, the pharmaceutically effective amount can be provided by including a plurality of the pharmaceutically active compounds on the derivatized collagen helix, e.g., by including large numbers of conjugated active compound per collagen helix an effective amount can be provided using fewer collagen helices, whereas in other examples the concentration of the derivatized collagen/conjugated compound can be selected to provide the effective amount.

In certain embodiments, the pharmaceutically active compound may be a non-synthetic pharmaceutically active compound whereas in other examples the pharmaceutically active compound may be a synthetic compound. Non-synthetic compounds include, but are not limited to, natural products and naturally occurring compounds. Synthetic compounds include analogs of non-synthetic pharmaceutically active compounds and other compounds not existing naturally or not yet found to exist naturally. Where a non-synthetic pharmaceutically active compound is conjugated to a derivatized collagen, the compound itself may be produced chemically, e.g., using total synthesis, even though the non-synthetic pharmaceutically active compound is naturally occurring. If desired, both a non-synthetic and synthetic pharmaceutically active compound may be conjugated to the derivatized collagen, e.g., to the methylated collagen.

In certain embodiments, the pharmaceutically active compound may be a protein, a carbohydrate, a lipid, a peptide, an amino acid, a nucleoside, a nitrogenous base, a nucleoside phosphate, an interference RNA (RNAi), a steroid, a high energy phosphate, a high energy biomolecule, an enzyme or other compounds, materials or components commonly present in one or more metabolic pathways of a cell.

In certain examples, the derivatized collagens described herein, e.g., methylated atelocollagens, can be conjugated to a growth factor such as, for example, adrenomedullin (AM), autocrine motility factor, a bone morphogenetic protein (BMP), a brain-derived neurotrophic factor (BDNF), an epidermal growth factor (EGF), erythropoietin (EPO), a fibroblast growth factor (FGF), a glial cell line-derived neurotrophic factor (GDNF), a granulocyte colony-stimulating factor (G-CSF), a granulocyte macrophage colony-stimulating factor (GM-CSF), a growth differentiation factor-9 (GDF9), a hepatocyte growth factor (HGF), a hepatoma-derived growth factor (HDGF), an insulin-like growth factor (IGF), a migration-stimulating factor, myostatin (GDF-8), a nerve growth factor (NGF) and other neurotrophins, a platelet-derived growth factor (PDGF), thrombopoietin (TPO), a transforming growth factor alpha(TGF-α), a transforming growth factor beta(TGF-β), a tumor necrosis factor-alpha (TNF-α), a vascular endothelial growth factor (VEGF), a Wnt Signaling Pathway protein, a placental growth factor (P1GF), a fetal bovine somatotrophin (FBS), IL-1- Cofactor for IL-3 and IL-6, IL-2- T-cell growth factor, IL-3, IL-4, IL-5, IL-6, IL-7 or other suitable growth factors. In some examples, any growth factor which can activate (or inhibit if desired) a kinase pathway, e.g., MAP kinase, PI3 kinase or the like, can be conjugated to the derivatized collagen. While the exact chemistry used to couple the growth factor to the derivatized collagen can vary, in many instances a free amino group of the growth factor can be coupled to a free carboxyl group of the collagen to provide an amide bond between the derivatized collagen and the growth factor. Where no such groups are present on the growth factor, the growth factor can be derivatized using one or more activating agents, e.g., a carbodiimide, a succinyl group, etc., and then reacted with a derivatized collagen.

In other configurations, the derivatized collagen can be conjugated to one or more therapeutics such as those described in Goodman and Gilman's the Pharmacological Basis of Therapeutics. Where a therapeutic is present, the therapeutic may be used alone as a pharmaceutically active compound or with another pharmaceutically active compound such as, for example, a growth factor or other selected active compound. Illustrative therapeutic agents include but are not limited to a neurotransmitter, a cholinase activator or inhibitor, an acetylcholinesterase activator or inhibitor, an acetylcholine, an acetylcholine agonist or derivative, a nicotinic receptor agonist, a nicotinic receptor antagonist, a muscarinic receptor agonist, a muscarinic receptor antagonist, dopamine, a dopamine derivative, a catecholamine, an adrenergic receptor agonist, an adrenergic receptor antagonist, an anticholinesterase agent, a nicotinic cholinergic receptor agonist, a nicotinic cholinergic receptor antagonist, a ganglionic blocking compound, a ganglionic stimulant, a serotonin receptor agonist, a serotonin receptor antagonist, an ion channel agonist, an ion channel antagonist, a neuromodulator, a therapeutic gas (e.g., oxygen, carbon dioxide, nitric oxide), ethanol or an ethanol derivative, a benzodiazepine analog, a phenothiazine, a thioxanthene, a heterocyclic compound, a sedative, a norepinephrine reuptake inhibitor, an antidepressant, a serotonin reuptake inhibitor, a monoamine oxidase agonist, a monoamine oxidase antagonist, a sodium ion channel activator, a sodium ion channel inhibitor, a calcium ion channel activator, a calcium ion channel inhibitor, a hydantoin, a barbituate, a stilbene, an iminostilbene, a succinimide, an oxazolidinedione, an antiseizure agent, an analgesic, an opioid, a peptide, an opioid agonist, an opioid antagonist, an autocoid, histamine, a histamine analog, a H1-receptor agonist, a H1-receptor antagonist, a H3-receptor agonist, a H3-receptor antagonist, an eicosanoid, a prostaglandin, a leukotriene, an anti-inflammatory agent, an antipyretic, a nonsteroidal anti-inflammatory agent, a salicylic acid derivative, a salicylate, a para-aminophenol derivative, an indole, an indene, an indole acetic acid, an indene acetic acid, a heteroaryl acetic acid, a propionic acid, an arylpropionic acid, an anthranilic acid, an enolic acid, an alkanone, an oxicam, gold and gold derivatives, a uricosuric agent, a corticosteroid, a bronchodilator, a diuretic, a vasopressin receptor agonist, a vasopressin receptor antagonist, angiotensin, an angiotensin analog, renin, a renin analog, an inhibitor of the renin-angiotensin system, an angiotensin receptor agonist, an angiotensin receptor antagonist, a renin inhibitor, an endopeptidase inhibitor, an organic nitrate, a calcium channel blocker, a beta-adrenergic receptor antagonist, an alpha -adrenergic receptor antagonist, an antiplatelet agent, an antithrombotic agent, an antihypertensive, a benzothiadiazine, a sympatholytic agent, a vasodilator, an angiotensin converting enzyme inhibitor, an angiotensin II receptor antagonist, a cardiac glycoside, a dopaminergic receptor agonist, a phosphodiesterase inhibitor, an antiarrhythmic drug, an HMG CoA reductase inhibitor, an H2 histamine receptor antagonist, an antibiotic, a hydrogen-potassium ATPase inhibitor, an antacid, a laxative, an antidiarrheal agent, an antiemetic agent, a prokinetic agent, oxytocin, an antimalarial agent, a diaminopyrimidine, quinine and quinine derivatives, quinoline and quinoline derivatives, an antihelminthic agent, an antimicrobial agent, a sulfonamide, a quinolone, a penicillin, a cephalosporin, a beta-lactam, an aminoglycoside, a tetracycline, a chloramphenicol, an erythromycin, an isonicotinic acid compound and derivatives thereof, a macrolide, a sulfone, an antifungal agent, an imidozole, a triazole, an antiviral agent, a protease inhibitor, an antiretroviral agent, a reverse transcriptase inhibitor, an acyclic nucleoside phosphonate, a nitrogen mustard, an ethylenimine, a methylmelamine, an alkyl sulfonate, a nitrosourea, a triazene, a folic acid analog, a pyrimidine analog, a purine analog, a vinca alkaloid, an epipodophyllotoxin, a coordination complex, a platinum coordination complex, an anthracenedione, a substituted urea, a methylhydrazine derivative, an adrenocortical suppressant, a progestin, an estrogen, an anti-estrogen, an androgen, an anti-androgen, a gonadotropin-releasing hormone analog, an immunosuppressant, an interferon, a granulocyte macrophage-colony stimulating factor, a tumor necrosis factor, an interleukin, an antibody, an antigen, a hematopoietic agent, an anticoagulant, a hormone, a growth hormone, a glucocorticoid, an antiseptic, insulin, a hypoglycemic agent, a hyperglycemic agent, an insulin analog, a vitamin, a water soluble vitamin, a fat soluble vitamin, a skin agent, an ocular agent, a cosmetic agent, a heavy metal antagonist, or other suitable synthetic or non-synthetic therapeutics. Where a therapeutic agent is present, it may be present alone or in combination with a biomolecule or another pharmaceutically active compound.

In some embodiments, the derivatized collagen described herein can include more than a single type of pharmaceutically active compound conjugated to it. For example, the derivatized collagen can first be reacted with a growth factor and subsequently reacted with a different pharmaceutically active compound to provide a hybrid derivatized collagen comprising more than a single type of pharmaceutically active compound.

In some embodiments, some portion of the derivatized collagen can be derivatized with a suitable active compound and the remainder of the derivatized collagen may not include any derivatized compound. For example, to control the particular level of one or more agents in an adhesive composition, a derivatized atelocollagen conjugated to a pharmaceutically active compound can be mixed with a derivatized atelocollagen lacking any pharmaceutically active compound. The two collagens or atelocollagens can be mixed or can be layered, e.g., the collagen comprising the active compound may be placed adjacent to the tissues or cells and then covered by the collagen lacking any active compound. Where layers of derivatized collagens are added, each layer can be cured before subsequent layers are added or the layers can first be built up with curing agent added only to the outer layer of the layered construct.

Crosslinking Agents and Conditions

In some examples, the crosslinking agents used herein may comprise one or more groups which can react with one or more amino acids (or other groups) present in the collagen or atelocollagen. For example, the collagen or atelocollagen typically comprises free amine groups (which may be N-terminal or present in one or more side chains), free carboxyl groups (which may be C-terminal or present in one or more side chains), free sulfhydryl groups (which are typically present in one or more side chains) or other reactive groups which can be derivatized. In some embodiments, two or more different crosslinking agents can be used so different groups present in the collagen or atelocollagen can be derivatized or one crosslinking agent may first react with the collagen or atelocollagen to form a first derivatized collagen or atelocollagen which then reacts with the second cross-linking agent to form a further derivatized collagen or atelocollagen.

In some embodiments, the cross-linking agent may comprise one or more groups including but not limited to, succinimidyl groups, hydroxysuccinimidyl groups, N-hydroxysuccinimdyl groups, sulfhydryl groups, amino groups and other suitable groups.

In some embodiments where two or more crosslinking agents are used one of the crosslinking agents may comprise one or more electrophilic groups and a second crosslinking agent may comprise one or more nucleophilic groups.

In some embodiments, the crosslinking agent(s) may be a synthetic polymer which includes one or more nucleophilic or electrophilic groups. For example, one or both of the crosslinking agents may comprise a synthetic polypeptide or an alkylene glycol, e.g., ethylene glycol, propylene glycol, polyethylene glycol, polypropylene glycol or other polyether based compounds, comprising one or more electrophilic groups or one or more nucleophilic groups. In some embodiments, one of the crosslinking agents used is a polyalkylene glycol comprising one or more electrophilic groups and the other crosslinking agent used is a polyalkylene glycol comprising one or more nucleophilic groups.

In some embodiments, one of the crosslinking agents may comprise a structure of formula (I)

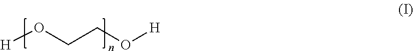

where n may vary between one to sixteen, for example, one to twelve, two to twelve, three to twelve, four to twelve, five to twelve, six to twelve, seven to twelve, eight to twelve, one to eight, two to eight, three to eight, four to eight, five to eight, six to eight, seven to eight, one to six, two to six, three to six, four to six, five to six, one to four, two to four, three to four, or one, two, three, four, five, six, seven, eight, nine, ten, eleven, twelve or other values less than sixteen. If desired, however, the number of chains comprising formula (I) can be greater than sixteen, e.g., up to 100 or more. In some embodiments, formula I may be present in a polyethylene glycol compound which is branched, star shaped, or has a comb geometry.

In other instances, compound comprising one or more of formulae (II)-(IV) may be used as a crosslinking agent.

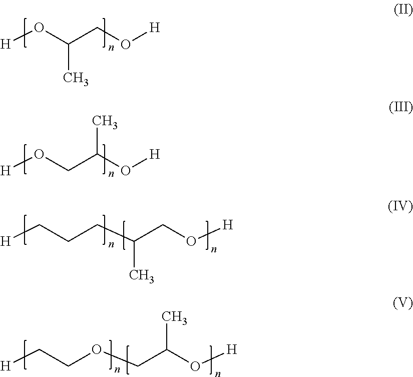

In any of formulae (II)-(V), n may be the same as the values described in connection with formula (I), e.g., two, three, four, five, six or more.

In certain embodiments, a compound including any of formula (I)-(V) may comprise at least one electrophilic group or at least one nucleophilic group. For example, one or more of formula (I)-(V) may comprise an electrophilic group(s) or a nucleophilic group(s). In some instances, a polyethylene glycol may be used that comprises one, two, three, four, five, six or more electrophilic groups or one, two, three, four, five, six or more nucleophilic groups. In some embodiments, each “arm” of the polyethylene glycol compound may comprise a respective nucleophilic or electrophilic group or at least one arm of a multi-arm polyethylene glycol compound may comprise a nucleophilic or electrophilic group. In certain instances, the polyethylene glycol may be a 2-armed PEG, a 4-armed PEG, an 8-armed PEG, a 12-armed PEG, a 16-armed PEG any one of which may comprise at least one nucleophilic or electrophilic group. In some embodiments, the groups present within a single PEG may be the same or may be different, e.g., different nucleophilic groups (or electrophilic groups) can be present on different arms of a PEG or each arm of a PEG may comprise the same nucleophilic groups (or electrophilic groups).

In some examples, the exact type and nature of the nucleophilic groups present in the crosslinking agent can vary and include, but are not limited to, -NH₂, -SH, -OH, —CO—NH—NH₂, etc. The number of nucleophilic groups present can vary from one to more than sixteen, e.g., one, two, three, four, five, six, seven, eight, nine, ten, eleven, twelve or more and may be the same or different. Similarly, the exact type and nature of the electrophilic groups present in the crosslinking agent can vary and include, but are not limited to succinimidyl, hydroxysuccinimidyl, N-hydroxysuccinimidyl, —CO₂—N(COCH₂)₂, -CO₂H, -CHO, -CHOCH₂, -N═C═O, —SO₂—CH═CH₂, -N(COCH)₂, —S—S—(C₅H₄N), etc., and may be the same or different. In certain embodiments, PEGs may be chemically modified to comprise primary amino or thiol groups according to known methods, e.g., Chapter 22 of Poly(ethylene Glycol) Chemistry: Biotechnical and Biomedical Applications, J. Milton Harris, ed., Plenum Press, N.Y. (1992). Various forms of modified PEGs are also commercially available from numerous sources.

In certain embodiments, multi-electrophilic polymers for use in with the collagens described herein may comprise two or more succinimidyl groups capable of forming covalent bonds with nucleophilic groups of the other crosslinking agent or the collagen. For example, succinimidyl groups can be highly reactive with materials containing primary amino (-NH₂) groups, such as multi-amino PEG, poly(lysine), or collagen. Succinimidyl groups are slightly less reactive with materials containing thiol (--SH) groups, such as multi-thiol PEG or synthetic polypeptides containing multiple cysteine residues, though the exact reactivity often depends on the particular reaction conditions including, for example, pH and temperature.

In some embodiments, one of the crosslinking agents described herein comprises at least one succinimidyl group and the other crosslinking agents comprises at least one amino group (primary or secondary) or at least one thiol (-SH) group. In other instances, one of the crosslinking agents comprises a two arm PEG comprising two succinimidyl groups and the other crosslinking agent comprises a two arm PEG comprising two of at least one amino group (primary or secondary) or at least one thiol (-SH) group, e.g., may comprise two amino groups, one amino group and one thiol group or two thiol groups. In other embodiments, one of the crosslinking agents comprises a four arm PEG comprising at least two succinimidyl groups, e.g., four succinimidyl groups, and the other crosslinking agent comprises a four arm PEG comprising two of at least one amino group (primary or secondary) or at least one thiol (-SH) group, e.g., may comprise two or more amino groups, at least one amino group and at least one thiol group or two or more thiol groups. Where the crosslinking agents each comprise a PEG, the PEGs may be the same armed PEGs, e.g., two 4-armed PEGs can be used, or may have different number of arms, e.g., one PEG may be a 4-armed PEG and the other PEG may be a 2-armed PEG or an 8-armed PEG.

In some embodiments, one or more crosslinking agents as described in any of U.S., Pat. Nos. 5,784,500, 6,312,725, 5,162,430, 5,151,689 or related continuation or continuation-in-part applications may be used. In some instances, at least two crosslinking agents from the aforementioned patents can be used with the derivatized collagens and in the processes described herein.

In certain examples, the crosslinking agents may be combined with each other and packaged in a separate vessel, e.g., a syringe, from the derivatized collagen. For example, the crosslinking agents may be combined and placed in a syringe under anhydrous conditions. The solid, sequestered curing agent can be added to the same syringe comprising the crosslinking agents. The derivatized atelocollagen can be present in a second syringe in a suspended solution form. The first syringe can be coupled to the second syringe and the collagen solution may be injected into the second syringe to combine the materials with each other. The materials may be permitted to cure in the second syringe for some period until a desired consistency is achieved. The materials can then be pushed out of the second syringe and placed onto a desired site optionally with the additional of more curing agent, which can be sequestered curing agent or non-sequestered curing agent or both.

Uses and Applications

The materials produced from the derivatized collagen, the crosslinking agents and the sequestered cuing agents can be used as an adhesive in biological and medical applications. As noted herein, the presence of atelocollagen can provide materials which are non-immunogenic or have reduced immunogenicity compared to native collagens. The adhesive may be designed to provide temporary adherence between two or more tissues to permit the tissues to undergo a repair process at the interface provided by the adhesive material. In some instances, the adhesive may be resorbed or degraded by the mammal during the repair process such that once the tissue is repaired, little or no adhesive remains behind. If desired, however, the adhesive composition may be selected such that a collagen framework (or other framework or structure) remains behind to provide reinforcement at the tissue repair site.

In certain embodiments, the materials described herein may be used to approximate two or more tissues to each other for at least some period. The tissues can be soft tissues, e.g., epithelial tissues, nervous tissue, etc. or may be “hard” tissues including bone, cartilage, tendon, ligament, etc. For example, the material can be applied to tissue tears or separations in skin, esophagus, lung tissue, ear tissues such as the tympanic membrane, the gastrointestinal tract (e.g., stomach, small intestine, large intestine, etc.), the reproductive tract, the urinary tract, exocrine gland tissue, endocrine gland tissue, the surface of the cornea, blood vessels, lymphatic vessels, the pericardium, the pleurae, the peritoneum and other epithelial tissues. The materials may be particularly effective at joining tissues in pulmonary tissues, blood vessels such as veins and arteries and the gastrointestinal tract. In the case of pulmonary tissue, the materials can repair tissues which are air tight under normal physiological pressure to permit proper functioning of the lungs. In the case of blood vessels, the material can repair tears which are liquid tight to permit proper circulatory pressure in veins or arteries. In the case of the gastrointestinal tract, the materials can repair tears to seal the intestinal lining to prevent intestinal material escaping into the abdomen.

In other instances, the materials described herein can be used to seal or repair tears or tissue damage or defects in muscle tissue including smooth muscle, skeletal muscle and cardiac muscle. To effectuate a repair, it may be desirable to isolate or immobilize the muscle tissue subsequent to curing of the defect with the materials described herein. For example, the adhesive materials described herein can be flexible and can stretch to some degree, but excessive movement of the muscle fibers could lead to tearing of the adhesive or the muscle fibers. If desired, to increase the overall flexibility and/or strength of the material elastomers, fibers or other materials can be mixed with the derivatized collagen materials prior to curing to alter the properties in a desired manner.

In additional examples, the materials described herein can be used to repair tears or defects in connective tissue including, for example, membranes, nerve coverings, meninges such as spinal cord meninges, connective tissues in the lymphatic system, white adipose tissue, brown adipose tissue, cartilage, bone and other types of connective tissues. If desired or needed, reinforcing materials such as fibers may be mixed with the derivatized collagen materials prior to curing to alter the properties in a desired manner.

In some examples, the materials described herein can be used as a wound adhesive or burn adhesive to treat defects or injuries in the skin. If desired, the materials can be mixed with stem cells to repair a wound, burn or other tissue defect. In additional instances, the derivatized collagen materials can be used in combination with a plurality of bone precursor cells, a plurality of cartilage precursor cells, or a plurality of epithelial cells. For example, the derivatized collagen materials can be mixed with precursor cells capable for forming into pancreatic cells, e.g., islet cells and/or Beta cells, to assist in the treatment of diabetes or other pancreatic conditions such as cancer. Similarly, the derivatized collagen materials can be used as a loading agent to load precursor cells of any mammalian organ and permit placement and retention of those cells at a desired site. In some instances, the precursor cells may be present in a layer or film below a cured layer or film of the derivatized collagen materials to permit the precursor cells to migrate into an area of the tissue. Growth factors, differentiation factors and the like may also be present with the precursor cells to assist the precursor cells in promoting tissue growth and/or in directing the precursor cells to differentiate into one or more desired cell or tissue types.

In certain embodiments, the adhesive strength provided by the materials described herein permits their use in bone injuries, bone damage and/or bone defects. For example, two or more bone pieces may be adhered to each other using the adhesives described herein in order to permit the bone defect to heal properly. In some instances, bone fragments, bone powder, demineralized bone powder or bone pieces can be mixed with the adhesive (optionally in combination with bone morphogenic proteins or other bone growth factors) and the mixture can be placed at a bone defect site to permit repair of the bone. In some instances, the bone fragment/adhesive mixture can be used to repair vertebral defects that may cause neck or spinal pain. In other instances, the adhesive may be placed at weak sites of bones, e.g., due to osteoporosis or other bone thinning disorders, to increase the overall strength of the bone at those sites. Such application may be performed by injecting the adhesive through a syringe at a desired bone site without the need for complicated surgical procedures.

In some embodiments, the adhesive can be applied to one piece of fractured bone and used to mend together two or more pieces of fractured, broken, shattered, etc. bone. In certain surgical procedures, holding pieces of bone together while drilling and mounting hardware is a difficult task. The adhesive material can be used as a temporary scaffold, designed to help hold the bone pieces together while a surgeon can add in the appropriate fasteners, such as screws, plates, etc. A permanent adhesive may hinder cellular movement into the bone, delaying healing.

In some embodiments, the adhesives may be loaded with or include one or more pharmaceutically active compounds. For example, hydrophobic pharmaceutically active compounds can be solubilized in hydrophobic domains of derivatized collagen of the adhesive with the collagen acting as reservoirs for such compounds. Where the adhesive is used as a delivery agent, the pharmaceutically active compound may not be covalently bound to the derivatized collagen but may instead be suspended or solubilized in the derivatized collagen. In some instances, the pharmaceutically active compound make take the form of a biological agent or compound, e.g., a peptide, protein, carbohydrate, lipid or the like, that can be conjugated to the derivatized collagens described herein. Illustrations of pharmaceutically active compounds are described in more detail below. In certain examples, a pharmaceutically active compound that can be delivered using the derivatized collagen can be a chemical compound, e.g., natural or synthetic, that can elicit a biological response, e.g., activate a cellular response or pathway, inhibit a cellular response or pathway, promote a cellular response or pathway, or alter a cellular response or pathway, under suitable conditions. For example, a pharmaceutically active compound can result in activation (or inactivation) of a cellular pathway, enzyme activation, enzyme inhibition, differentiation of a stem cell into a desired cell type, repair of a tissues, cellular enhancement, inhibition of cellular processes, activation (or deactivation) of an ion channel, an increase (or decrease) in protein expression, an increase (or decrease) in RNA production, an increase (or decrease) in mitosis, an increase (or decrease) in meiosis, an increase (or decrease) in intracellular transport, or other activities that a cell may perform or initiate through one or more cellular systems or pathways.

In certain examples, when a pharmaceutically active compound is present in a mixture with the adhesive, it may be present in an effective amount, e.g., an amount effective to elicit the biological response. In some configurations, the concentration of pharmaceutically active compound may exceed the amount desired to elicit a biological response such that sustained release of the pharmaceutically active compound can provide for such biological response for extended periods. It will be recognized by the person of ordinary skill in the art, given the benefit of this disclosure, that there are multiple ways to provide an effective amount of the pharmaceutically active compound.

In certain embodiments, the pharmaceutically active compound to be delivered may be a non-synthetic pharmaceutically active compound whereas in other examples the pharmaceutically active compound to be delivered may be a synthetic compound. Non-synthetic compounds include, but are not limited to, natural products and naturally occurring compounds. Synthetic compounds include analogs of non-synthetic pharmaceutically active compounds and other compounds not existing naturally or not yet found to exist naturally. Where a non-synthetic pharmaceutically active compound is delivered using a derivatized collagen, the compound itself may be produced chemically, e.g., using total synthesis, even though the non-synthetic pharmaceutically active compound is naturally occurring. If desired, both a non-synthetic and synthetic pharmaceutically active compound may be delivered using a derivatized collagen, e.g., to the methylated collagen.

In certain embodiments, the pharmaceutically active compound that can be delivered using an adhesive may be a protein, a carbohydrate, a lipid, a peptide, an amino acid, a nucleoside, a nitrogenous base, a nucleoside phosphate, an interference RNA (RNAi), a steroid, a high energy phosphate, a high energy biomolecule, an enzyme or other compounds, materials or components commonly present in one or more metabolic pathways of a cell.

In certain examples, the adhesives described herein, e.g., methylated atelocollagens, can be used to deliver a growth factor such as, for example, adrenomedullin (AM), autocrine motility factor, a bone morphogenetic protein (BMP), a brain-derived neurotrophic factor (BDNF), an epidermal growth factor (EGF), erythropoietin (EPO), a fibroblast growth factor (FGF), a glial cell line-derived neurotrophic factor (GDNF), a granulocyte colony-stimulating factor (G-CSF), a granulocyte macrophage colony-stimulating factor (GM-CSF), a growth differentiation factor-9 (GDF9), a hepatocyte growth factor (HGF), a hepatoma-derived growth factor (HDGF), an insulin-like growth factor (IGF), a migration-stimulating factor, myostatin (GDF-8), a nerve growth factor (NGF) and other neurotrophins, a platelet-derived growth factor (PDGF), thrombopoietin (TPO), a transforming growth factor alpha(TGF-α), a transforming growth factor beta(TGF-β), a tumor necrosis factor-alpha (TNF-α), a vascular endothelial growth factor (VEGF), a Wnt Signaling Pathway protein, a placental growth factor (P1GF), a fetal bovine somatotrophin (FBS), IL-1- Cofactor for IL-3 and IL-6, IL-2- T-cell growth factor, IL-3, IL-4, IL-5, IL-6, IL-7 or other suitable growth factors. In some examples, any growth factor which can activate (or inhibit if desired) a kinase pathway, e.g., MAP kinase, PI3 kinase or the like, can be delivered using a derivatized collagen.

In other configurations, the adhesive can be used to deliver one or more therapeutics such as those described in Goodman and Gilman' s the Pharmacological Basis of Therapeutics. Where a therapeutic is present, the therapeutic may be used alone as a pharmaceutically active compound or with another pharmaceutically active compound such as, for example, a growth factor or other selected active compound. Illustrative therapeutic agents include but are not limited to a neurotransmitter, a cholinase activator or inhibitor, an acetylcholinesterase activator or inhibitor, an acetylcholine, an acetylcholine agonist or derivative, a nicotinic receptor agonist, a nicotinic receptor antagonist, a muscarinic receptor agonist, a muscarinic receptor antagonist, dopamine, a dopamine derivative, a catecholamine, an adrenergic receptor agonist, an adrenergic receptor antagonist, an anticholinesterase agent, a nicotinic cholinergic receptor agonist, a nicotinic cholinergic receptor antagonist, a ganglionic blocking compound, a ganglionic stimulant, a serotonin receptor agonist, a serotonin receptor antagonist, an ion channel agonist, an ion channel antagonist, a neuromodulator, a therapeutic gas (e.g., oxygen, carbon dioxide, nitric oxide), ethanol or an ethanol derivative, a benzodiazepine analog, a phenothiazine, a thioxanthene, a heterocyclic compound, a sedative, a norepinephrine reuptake inhibitor, an antidepressant, a serotonin reuptake inhibitor, a monoamine oxidase agonist, a monoamine oxidase antagonist, a sodium ion channel activator, a sodium ion channel inhibitor, a calcium ion channel activator, a calcium ion channel inhibitor, a hydantoin, a barbituate, a stilbene, an iminostilbene, a succinimide, an oxazolidinedione, an antiseizure agent, an analgesic, an opioid, a peptide, an opioid agonist, an opioid antagonist, an autocoid, histamine, a histamine analog, a H1-receptor agonist, a H1-receptor antagonist, a H3-receptor agonist, a H3-receptor antagonist, an eicosanoid, a prostaglandin, a leukotriene, an anti-inflammatory agent, an antipyretic, a nonsteroidal anti-inflammatory agent, a salicylic acid derivative, a salicylate, a para-aminophenol derivative, an indole, an indene, an indole acetic acid, an indene acetic acid, a heteroaryl acetic acid, a propionic acid, an arylpropionic acid, an anthranilic acid, an enolic acid, an alkanone, an oxicam, gold and gold derivatives, a uricosuric agent, a corticosteroid, a bronchodilator, a diuretic, a vasopressin receptor agonist, a vasopressin receptor antagonist, angiotensin, an angiotensin analog, renin, a renin analog, an inhibitor of the renin-angiotensin system, an angiotensin receptor agonist, an angiotensin receptor antagonist, a renin inhibitor, an endopeptidase inhibitor, an organic nitrate, a calcium channel blocker, a beta-adrenergic receptor antagonist, an alpha -adrenergic receptor antagonist, an antiplatelet agent, an antithrombotic agent, an antihypertensive, a benzothiadiazine, a sympatholytic agent, a vasodilator, an angiotensin converting enzyme inhibitor, an angiotensin II receptor antagonist, a cardiac glycoside, a dopaminergic receptor agonist, a phosphodiesterase inhibitor, an antiarrhythmic drug, an HMG CoA reductase inhibitor, an H2 histamine receptor antagonist, an antibiotic, a hydrogen-potassium ATPase inhibitor, an antacid, a laxative, an antidiarrheal agent, an antiemetic agent, a prokinetic agent, oxytocin, an antimalarial agent, a diaminopyrimidine, quinine and quinine derivatives, quinoline and quinoline derivatives, an antihelminthic agent, an antimicrobial agent, a sulfonamide, a quinolone, a penicillin, a cephalosporin, a beta-lactam, an aminoglycoside, a tetracycline, a chloramphenicol, an erythromycin, an isonicotinic acid compound and derivatives thereof, a macrolide, a sulfone, an antifungal agent, an imidozole, a triazole, an antiviral agent, a protease inhibitor, an antiretroviral agent, a reverse transcriptase inhibitor, an acyclic nucleoside phosphonate, a nitrogen mustard, an ethylenimine, a methylmelamine, an alkyl sulfonate, a nitrosourea, a triazene, a folic acid analog, a pyrimidine analog, a purine analog, a vinca alkaloid, an epipodophyllotoxin, a coordination complex, a platinum coordination complex, an anthracenedione, a substituted urea, a methylhydrazine derivative, an adrenocortical suppressant, a progestin, an estrogen, an anti-estrogen, an androgen, an anti-androgen, a gonadotropin-releasing hormone analog, an immunosuppressant, an interferon, a granulocyte macrophage-colony stimulating factor, a tumor necrosis factor, an interleukin, an antibody, an antigen, a hematopoietic agent, an anticoagulant, a hormone, a growth hormone, a glucocorticoid, an antiseptic, insulin, a hypoglycemic agent, a hyperglycemic agent, an insulin analog, a vitamin, a water soluble vitamin, a fat soluble vitamin, a skin agent, an ocular agent, a cosmetic agent, a heavy metal antagonist, or other suitable synthetic or non-synthetic therapeutics. Where a therapeutic agent is present, it may be present alone or in combination with a biomolecule or another pharmaceutically active compound.

In some embodiments, the adhesives described herein can be used to deliver more than a single type of pharmaceutically active compound. For example, the derivatized collagen can be mixed with a growth factor and a different pharmaceutically active compound to provide a three component mixture.

In certain embodiments, the adhesives described herein can be mixed with one or more pharmaceutically active agents or biologically active agents and used, at least in part, to deliver these agents to a specific tissue site. For example, the adhesives can be mixed with one or more pharmaceutically active agents or biologically active agents prior to curing. The resulting combination may be disposed at a desired site to permit the agent to be in proximity to the site. The material may then be fully cured as described herein. In some embodiments where the collagen is mixed with a pharmaceutically active agent or biologically active agent, it may be desirable to layer the material to provide some uncured or partially cured material closest to the tissue site and a cured covering or cured cover layer to seal the pharmaceutically active agents or biologically active agents underneath. By keeping the layer adjacent to the tissue at least partially fluid, the pharmaceutically active agents or biologically active agents can diffuse into the tissue and provide some therapeutic effect.

In certain embodiments, the various components used to produce the materials described herein may be present in kit form. For example, the kit may comprise a first container or vessel which includes a derivatized atelocollagen. In a second vessel or container, one or more of a first crosslinking agent, a second crosslinking agent different from the first crosslinking agent, and a sequestered curing agent may be present. The kit may also include instructions for using the derivatized atelocollagen, the first crosslinking agent, the second crosslinking agent and the sequestered curing agent to provide an adhesive material. In some instances the derivatized atelocollagen of the kit comprises a number average molecular weight of less than 300 kDa. In certain examples, the first crosslinking agent of the kit comprises a functionalized polyethylene glycol crosslinking agent. In other examples, the second crosslinking agent of the kit comprises a functionalized polyethylene glycol crosslinking agent different from the first crosslinking agent. In some embodiments, each of the first crosslinking agent and the second crosslinking agent of the kit is independently selected from the group consisting of an N-hydroxysuccinimidyl functionalized polyethylene glycol, a sulfhydryl functionalized polyethylene glycol, and an amino functionalized polyethylene glycol, and in which the first crosslinking agent is different than the second crosslinking agent. In some embodiments, the sequestered curing agent may comprise calcium, e.g., calcium alginate which may be present as a coacervate, agglomerate or encapsulate. In some instances, the sequestered curing agent comprises one or more of a bicarbonate compound, a carbonate compound, a phosphate compound, a sodium compound, a potassium compound, a calcium compound, and combinations thereof. In certain embodiments, the sequestered curing agent does not comprise any glutaraldehyde. In some examples, the sequestered curing agent is present in solid form and may optionally be anhydrous. In certain examples, the collagen is present in a solution or a suspension.

In other configurations of a kit, the kit may comprise collagen comprising an atelocollagen, a derivatization agent, a first crosslinking agent, a second crosslinking agent different from the first crosslinking agent, a sequestered curing agent, instructions for using the atelocollagen and derivatization agent to provide a derivatized atelocollagen, and instructions for using the provided derivatized atelocollagen, the first crosslinking agent, the second crosslinking agent and the curing agent to provide an adhesive material. If desired, components and instructions needed to produce the sequestered curing agent could instead be present in place of (or in addition to) the sequestered curing agent. In some configurations, the derivatized atelocollagen comprises a number average molecular weight of less than 300 kDa. In other configurations, the first crosslinking agent comprises a functionalized polyethylene glycol crosslinking agent. In further configurations, the second crosslinking agent comprises a functionalized polyethylene glycol crosslinking agent different from the first crosslinking agent. In some examples, each of the first crosslinking agent and the second crosslinking agent is independently selected from the group consisting of an N-hydroxysuccinimidyl functionalized polyethylene glycol, a sulfhydryl functionalized polyethylene glycol, and an amino functionalized polyethylene glycol, in which the first crosslinking agent is different than the second crosslinking agent. In some examples, the sequestered curing agent is present as a solid coacervate, a solid agglomerate or solid encapsulated particles. In other embodiments, the curing agent comprises one or more of a bicarbonate compound, a carbonate compound, a phosphate compound, a sodium compound, a potassium compound, a calcium compound and combinations thereof. In some instances, the curing agent does not comprise any glutaraldehyde. In further examples, the kit may comprise instructions for using the curing agent in solid form to provide the adhesive material. In certain embodiments, the collagen is present in a solution or in a suspension.

Certain specific test results are described below to illustrate further some of the conditions and uses of the materials described herein.

### EXAMPLE 1

Solid atelocollagen obtained from porcine hide (from Nippon Ham, Japan) was methylated using the following two-step process: (1) the solid atelocollagen was placed in a solution of methanol/hydrochloric acid (a solution prepared in methanol containing 0.9 M diethyl ether and 0.1 M hydrochloric acid) at room temperature for 72 hours, and (2) the resulting product was washed 3X with ethanol. After washing, the methylated atelocollagen was vacuum dried for 18 hours. No further washing steps or any washing steps with an aqueous solution were performed prior to use.

The resulting methylated atelocollagen was about 90% methylated (as verified using a colorimetric spectroscopic method using a hydroxylamine compound and the number of carboxyl groups calculated present in the atelocollagen) and could be loaded at a level of about 38 mg/mL (as determined by BCA analysis) to provide a desired viscosity. The number average molecular weight of the methylated collagen was 300 kDa or less. A higher quality collagen (one containing higher amounts of monomeric collagen vs dimer/trimeric) has a lower viscosity for a selected concentration. This permits loading of the collagen at a higher level (e.g., 38 mg/mL) to provide a desired viscosity.

A native PAGE gel comparing the methylated atelocollagen with non-methylated atelocollagen shows that the methylated atelocollagen migrated further than the non-methylated atelocollagen. This result is consistent with the methyl group forming an ester with carboxylic acid groups and removing the negative charge from those groups, which provides a more overall positive charge for the methylated atelocollagen molecules.

### EXAMPLE 2

A sequestered curing agent was used with the methylated atelocollagen produced in Example 1 and was prepared as microparticles. To prepare microparticles, a 9 mg/ml solution of sodium alginate was prepared in deionized water. A curing component, sodium carbonate, was added to the alginate solution (3.6 mg sodium carbonate/ml alginate solution) and mixed until completely dissolved. The alginate solution was then added to an airbrush sprayer. The airbrush was attached to a nitrogen gas cylinder and the pressure set to 20-22 psi. The alginate solution was then sprayed into a beaker of calcium chloride solution (1 M) with stirring. Upon contact with the calcium chloride solution, the small particles of alginate solution ionotropically gelled to form alginate microspheres. The mixture of solvent and microspheres was then centrifuged at 4,000g for 30 minutes. The supernatant (excess solvent) was decanted and the pellet (microspheres) were added to a small petri dish and left to air dry overnight in a fume hood.

### EXAMPLE 3

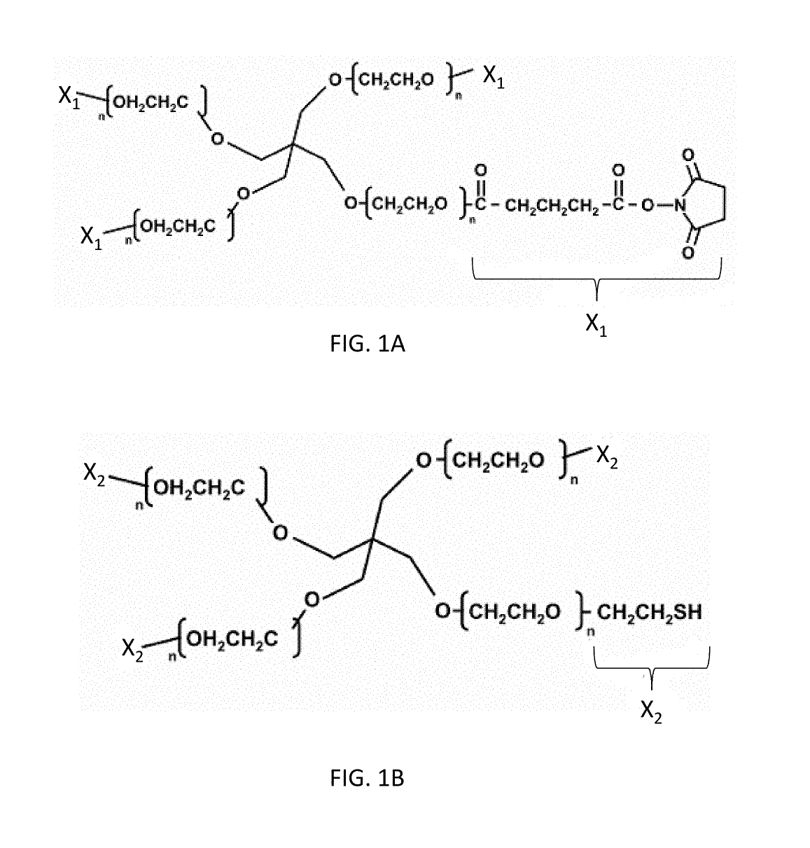

The methylated atelocollagen produced in Example 1 was used in combination (at a concentration of 39 mg/mL) with the microparticle sequestered curing agent produced in Example 2 and was crosslinked in the presence of two crosslinking agents. The methylated atelocollagen was present along with a solution at a pH of about 3.5-4.1 in a first syringe. Two crosslinking agents (one 4-arm NHS-PEG (see FIG. 1A), one 4-arm SH-PEG (see FIG. 1B)) purchased commercially from NOF America Corp. (White Plains, NY)), and the microparticles of Example 2 were placed together as powders in a second syringe. The acidic methylated atelocollagen was injected from the first syringe into the second syringe to mix the components. Any air bubbles were removed/minimized from the syringe.

The addition of alginate curing agent microparticles lowered the solution viscosity of solution mixture significantly—the normal solution mixture has a thick, “toothpaste-like” viscosity, while the addition of microparticle curing agent lowered the viscosity to a thinner solution, similar to warm honey.

### EXAMPLE 4

The freshly mixed material combination from Example 3 was then dispersed onto a piece of sausage casing, which had been blotted dry with surgical gauze. The low viscosity fluid nature of the produced adhesive allowed it to flow and be easily spread across the surface, which may assist in improving adhesion by filling in any defects or imperfections in tissue surfaces. Once a thin coating was achieved on the surface, a second piece of sausage casing (also blotted dry) was laid on top of the disposed adhesive. The two pieces were pressed together to assure firm contact. The casing was then left undisturbed to allow curing to take place. After 14 minutes of curing, the top piece of sausage casing was adhered to the bottom piece. Forceps were used to peel off the top layer, however with great difficulty. In some regions, portions of the sausage casing had begun to tear, indicating strong adhesion.

### EXAMPLE 5

Various mechanical tests using the material from Example 3 were performed using an Instron 3300 Single Column Universal Testing System. The shear strength of the material was measured to be about 57 N on average (varied from 30-72N) using the ASTM 2255 test dated 2003. The T-peel strength of the material was measured to be 0.23 N with a peak value of 0.75N as tested by the ASTM 2256 test dated 2005. In comparison, published literature values for TissuGlu® surgical adhesive (polyurethane based adhesive) are 32.6 N for the shear strength as tested by ASTM 2255 and 0.36-0.92N for the T-peel strength as tested by ASTM 2256. These results are consistent with the collagen based material providing similar or better results than the polyurethane based material.

### EXAMPLE 6

The adhesive material and curing agent are applied to one piece of fractured bone and used to mend together two or more pieces of fractured, broken, shattered, etc. bone. The adhesive can be used as temporary scaffold, designed to help hold the bone pieces together while a surgeon can add in the appropriate fasteners, such as screws, plates, etc.

### EXAMPLE 7

The adhesive material and curing agent is applied to at least one tissue at a seroma site to hold the two tissues together and prevent re-accumulation of fluid between the two tissue layers. For example, fluid may be drained from the seroma site, and then the adhesive material and curing agent can be introduced to hold the tissues together and prevent future fluid accumulation at that site.

### EXAMPLE 8

A mixture of mammalian, predominately Type I, either dermis or tendon-derived ,predominantly monomeric atelocollagen in dry form (as opposed to solution) from bovine or porcine sources is added to a solution of 0.9 M diethyl ether and 0.1 M hydrochloric acid prepared in methanol (methylation solution) at a concentration of 2% (w/w) and allowed to react for 72 hours. The collagen is generally in a granular particle form but powders, grains, pellets or other solid shapes are also suitable.

Following methylation, the methylation solution is decanted off and the methylated atelocollagen is washed and filtered three times using absolute ethanol, 10 minutes for each wash. The methylated atelocollagen is then placed under vacuum for 18 hours to remove any remaining solvent, producing dry methylated atelocollagen.

Once dried, the material is added to an aqueous buffered solution (pH 3.5) to produce a desired final collagen concentration (e.g., between 25-60 mg/mL or 50-70 mg/mL), as required and/or desired by the specific application. As noted herein, the material can be used and cured using one or more sequestered curing agents.

When introducing elements of the aspects, embodiments and examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

1. An adhesive material comprising a crosslinked, derivatized atelocollagen and a sequestered curing agent.

2. The adhesive material of claim 1, in which the derivatized atelocollagen is an alkylated mammalian atelocollagen or a methylated atelocollagen, in which the alkylated or methylated derivatized atelocollagen comprises:
a number average molecular weight of about 300 kDa; or
a number average molecular weight of about 300 kDa to about 600 kDa; or
a number average molecular weight of less than 300 kDa.

3. The adhesive material of claim 1, in which the sequestered curing agent is a solid curing agent.

4. The adhesive material of claim 1, in which the crosslinked, derivatized atelocollagen is crosslinked with a polyethylene glycol crosslinking agent.

5. The adhesive material of claim 4, in which the polyethylene cros slinking agent comprises at least one of a succinimidyl group, a sulfhydryl group or an amino group.

6. The adhesive material of claim 4, in which the polyethylene glycol cros slinking agent comprises a first crosslinking agent comprising at least one electrophilic group and a second crosslinking agent comprising at least one nucleophilic group.

7. The adhesive material of claim 1, in which the crosslinked, derivatized atelocollagen is cured at a curing time of more than 10 minutes to provide the adhesive material.

8. The adhesive material of claim 1, in which the derivatized atelocollagen is methylated mammalian atelocollagen, in which a first crosslinking agent comprises the formula of FIG. 1A and a second crosslinking agent comprises the formula of FIG. 1B.

9. The adhesive material of claim 8, in which the sequestered curing agent comprises divalent metal ions in combination with a sequestering agent and in which the sequestered curing agent is present in solid form.

10. The adhesive material of claim 9, in which the sequestering agent is selected from the group consisting of an alginate, a polysaccharide, hyaluronic acid, a glycosoaminoglycan and combinations thereof.

11. An adhesive material comprising a crosslinked, derivatized atelocollagen that has been cross-linked in the presence of at least two different functionalized crosslinking agents and an encapsulated curing agent.

12. The adhesive material of claim 11, in which the derivatized atelocollagen is a methylated porcine atelocollagen comprising a number average molecular weight of about 300 kDa.

13. The adhesive material of claim 11, in which the crosslinked, derivatized atelocollagen is cured with solid, encapsulated curing agent.

14. The adhesive material of claim 11, in which at least one of the different functionalized crosslinking agents comprises a polyethylene glycol functionalized with an electrophilic group.

15. The adhesive material of claim 14, in which the polyethylene crosslinking agent functionalized with an electrophilic group comprises at least one of a succinimidyl group.

16. The adhesive material of claim 14 in which another one of the different functionalized crosslinking agent agents comprises a polyethylene glycol functionalized with a nucleophilic group.

17. The adhesive material of claim 16, in which the polyethylene crosslinking agent functionalized with a nucleophilic group comprises at least one of a sulfhydryl group or an amino group.

18. The adhesive material of claim 11, in which the derivatized atelocollagen is methylated porcine atelocollagen, in which a first crosslinking agent of the two different functionalized crosslinking agents comprises the formula of FIG. 1A and a second crosslinking agent of the two different functionalized crosslinking agents comprises the formula of FIG. 1B.

19. The adhesive material of claim 11, in which the sequestered curing agent comprises divalent metal ions in combination with a sequestering agent and in which the sequestered curing agent is present in solid form.

20. The adhesive material of claim 19, in which the sequestering agent is selected from the group consisting of an alginate, a polysaccharide, hyaluronic acid, glycosoaminoglycan and combinations thereof.

21-194. (canceled)
